(19) United States
(12) Patent Application Publication
(10) Pub. No.: US 2008/0318333 A1
(43) Pub. Date: Dec. 25, 2008

(54) Method and System for Measurement of Nitrite and Nitric Oxide Release

(75) Inventors: Kent Hoier Nielsen, Olstykke (DK); Lars Niklas Larsson, Lund (SE)

Correspondence Address: Muncy, Geissler, Olds & Lowe, PLLC, P.O. BOX 1364, FAIRFAX, VA, 22038-1364, US

(73) Assignee: MILLIMED A/S, Roskilde (DK)

(21) Appl. No.: 12/097,151

(22) PCT Filed: Dec. 13, 2006
(86) PCT No.: PCT/DK2006/000714

§ 371 Date: Jul. 16, 2008

Related U.S. Application Data
(60) Provisional application No. 60/749,602, filed on Dec. 13, 2005.

(30) Foreign Application Priority Data
Dec. 13, 2005 (EP) 05027187.3

(51) Int. Cl. G01N 33/00

(52) U.S. Cl. 436/110

(57) ABSTRACT

A method for combined measurement of release of nitrite (NO₂⁻) and nitric oxide (NO) from a probe containing nitric oxide, such as an intravascular medical device immersed in an aqueous solution within a first vessel, comprises conveying nitric oxide (NO) released directly from the probe to a nitric oxide analysis apparatus. Nitrite is removed from a first vessel, which may e.g. comprise a head-space chamber, and transferred to a purge vessel where NO₂⁻ is transformed to nitric oxide (NO), which is conveyed to the nitric oxide analysis apparatus. A selector valve arranged upstream of the nitric oxide analysis apparatus is operated to selectively allow one of the directly released nitric oxide (NO) and the nitrite-derived nitric oxide (NO) into the apparatus. Directly released nitric oxide may be continuously flushed away from the first vessel.

### TECHNICAL FIELD

The present invention relates to methods and system for measurement of nitrite (NO₂⁻) and nitric oxide (NO). In particular, the invention is concerned with the use of a nitric oxide analysis apparatus for the continuous measurement of nitric oxide released from a probe and for intermittent measurement of nitrite released from the same probe.

### BACKGROUND OF THE INVENTION

Yang Fan et al. 1997 (Clinical Chemistry, vol 43:4, pp 657-662) refers to the effects of reducing reagents and temperature on conversion of known amounts of nitrite to nitric oxide and detection by NO by chemiluminescence, and provides a commonly applied method for the conversion of nitrite to nitric oxide based on conversion by an acetic acid-sodium iodide mixture.

Careri et al., 1999 (J. of Chromatography 848:1-2, pp 327-335) refers to the evaluation of dynamic headspace and purge trap techniques for the high resolution gas-chromatography analysis of nitric oxide in seawater.

U.S. Pat. No. 4,412,006 refers to a technique of determination of nitrate-nitrite content of a test sample, without also determining additional nitrogen content. The nitrate-nitrite content of a sample is reduced to nitric oxide which is determined via its chemiluminescence reaction with ozone. Nitrite is selectively reduced under mild conditions and the total nitrate-nitrite content is determined by stronger reduction conditions.

Nitric oxide (NO) has proven to be a useful agent in a wide range of physiological processes, such as reendothelialization, vasodilation, neurotransmission and platelet aggregation. The biological function of NO has also been found to include action as cytotoxic agent. Therefore, the need for studying NO release from biological and chemical molecules has increased, e.g. for the purpose of simulating NO release from medical devices during research and development of such devices. However, NO readily reacts with oxygen and water, forming nitrite (NO₂⁻) which acts as an interfering molecule in most available methods used for measuring NO.

### SUMMARY OF THE INVENTION

It is thus an object of preferred embodiments of the present invention to provide a method and a system capable of obtaining an NO measurement, in which the presence of nitrite can be taken into account or even eliminated in order to obtain a more precise NO measurement than hitherto achievable. It is a further object of preferred embodiments of the invention to provide a method and a system facilitating the procedure of obtaining reliable measurements of NO, or both NO and NO₂⁻.

The invention provides a system for (combined) measurement of the release of nitrite and nitric oxide from a probe containing nitric oxide, such as (in the form of) a nitric oxide adduct, the system comprising:
a first vessel for accommodation of the probe;
a nitric oxide analysis apparatus;
a gas feed conduit, through which nitric oxide may be conveyed to said analysis apparatus;
a first conduit, through which nitric oxide released directly from the probe may be conveyed from the first vessel to an upstream end of the gas feed conduit;
a purge vessel arranged upstream of a second conduit, whereby nitrite may be transformed to nitric oxide in the purge vessel, the second conduit being arranged to convey nitrite-derived nitric oxide to the upstream end of the gas feed conduit;
a selector valve arranged at the upstream end of the gas feed conduit, the selector valve being connected to respective downstream ends of the first and second conduits, and arranged to selectively allow directly released nitric oxide and nitrite-derived nitric oxide into the gas feed conduit.

Thanks to the provision of a selector valve, NO release and NO₂⁻ background can be monitored in a single analytical run. Following NO gas and liquid nitrite calibrations of the nitric oxide analysis apparatus, an output mV signal of the apparatus will be indicative of actual concentrations.

The present invention thus provides a method for (combined) measurement of the release of nitrite (NO₂⁻) and nitric oxide (NO) from a probe containing nitric oxide, such as a nitric oxide adduct, comprising the steps of:
placing the probe in a first vessel, in which nitrite (NO₂⁻) and nitric oxide (NO) is released from the probe;
conveying nitric oxide (NO) released (directly) from the probe through a first conduit to a gas feed conduit, the gas feed conduit being arranged to convey nitric oxide into a nitric oxide analysis apparatus;
removing a nitrite (NO₂⁻) sample from the first vessel;
transforming the nitrite (NO₂⁻) in the removed nitrite sample to nitric oxide (NO) to obtain nitrite-derived nitric oxide, the step of transforming occurring subsequent to the step of removing nitrite from the first vessel;
conveying the nitrite-derived nitric oxide through a second conduit to the gas feed conduit;
operating a selector valve arranged at an upstream end of the gas feed conduit and at respective downstream ends of the first and second conduits, so as to selectively allow one of the directly released nitric oxide (NO) and the nitrite-derived nitric oxide (NO) into the gas feed conduit.

The amount of directly released nitric oxide (NO) and the nitrite-derived nitric oxide (NO) present in a sample may advantageously be determined using suitable analytical methods, such as those referred to herein.

The present invention thus provides a method for the measurement of the release of nitrite (NO₂⁻) and nitric oxide from a sample, comprising the following steps:

a) Obtaining at least two equivalent fractions of a sample comprising a combined mixture of nitrite and nitric oxide;
c) determining the concentration of nitric oxide released from a first fraction;
d) converting the nitrite component of a second fraction to nitric oxide;
e) determining the concentration of nitric oxide released from the second fraction after conversion of nitrite to nitric oxide;
f) comparing the levels of nitric oxide determined in step (c) and step (e) to determine the concentration of nitrite (NO₂⁻) and nitric oxide in the sample
wherein steps c) and d) may be carried out in any order.

The above method for the combined measurement of the release of nitrite (NO₂⁻) and nitric oxide from a sample may, of course be performed using the system according to the invention, and as such may comprise part of the method for combined measurement of release of nitrite (NO₂⁻) and nitric oxide (NO) from a probe. It will be apparent therefore that the specific features of each of these aspects of the invention, as described herein, may, where appropriate refer to the other aspects.

In a particularly preferred embodiment, the method(s) and system of the present invention are suitable for studying quantitative on-line release of NO combined with quantitative measurements of nitrite (NO₂⁻), using a combined setup of a dynamic head space and purge vessel system enabling precise quantitative measurements of NO release and NO₂⁻ concentrations from the same probe.

The nitric oxide analysis apparatus may comprise an apparatus known per se, for example a so-called high-sensitivity detector for measuring nitric oxide based on a gas-phase chemiluminescent reaction between nitric oxide and ozone:

NO+O₃->NO₂*+O₂

NO₂*->NO₂+hv

Emission from electronically excited nitrogen dioxide is in the red and near-infrared region of the spectrum, and may be detected by a thermoelectrically cooled, red-sensitive photomultiplier tube.

The sensitivity of the photomultiplier, and thereby the detectable concentration range of nitric oxide, can be controlled by controlling the voltage supplied to the photomultiplier, preferably by using a potentiometer.

One suitable analysis apparatus is the Nitric Oxide Analyzer NOA™ 280i commercially available from Sievers®, Boulder, Colo., USA.

### DETAILED DESCRIPTION OF THE INVENTION

Preferred embodiments of method and system of the present invention will now be described with reference to the accompanying drawings, in which:

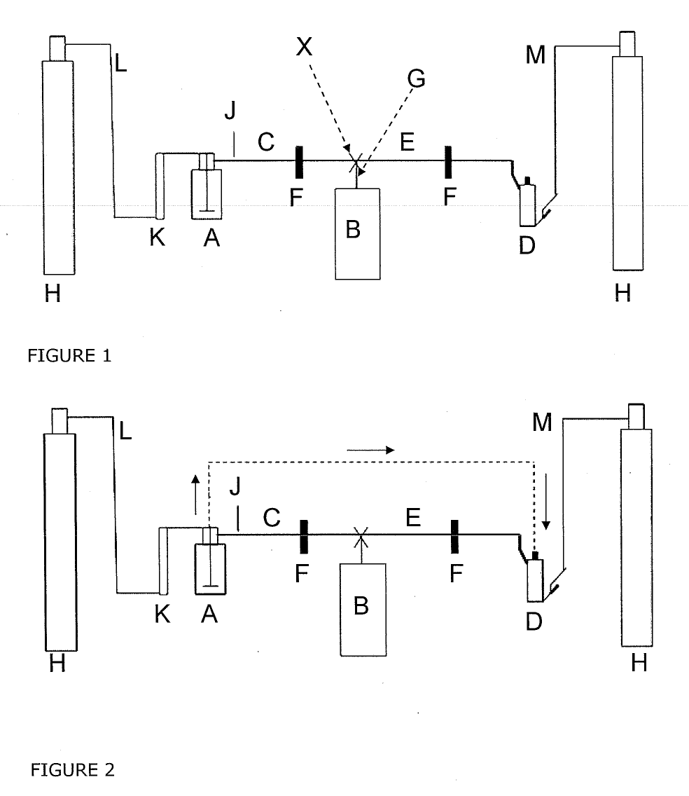

FIGS. 1, 2 & 3 show, in schematic illustration, three embodiments of the system according to the invention, where a combined setup of (e.g) a dynamic head space and purge vessel system enabling precise and quantitative measurements of NO release and NO₂⁻ concentrations from the same probe during the same analytic procedure.

Key to FIGS. 1, 2 and 3: A: First vessel (e.g. head space chamber), B: NO analyzer apparatus, C: First conduit, D: Purge vessel, E: Second conduit, X: Selector valve, F: Filter, G: Gas feed conduit, H: Inert gas source, J: Vent (e.g. T connection for release of gas), K:

Flow controller and/or flow meter, L: First inert gas conduit, M: Second Inert gas conduit.

FIG. 1 shows a system according to the invention wherein the first vessel (A) and the purge vessel (D) are in series.

FIG. 2 illustrates the process of transfer of a nitrite sample (a sample which comprises nitrite) from the first vessel (A) to the purge vessel (D).

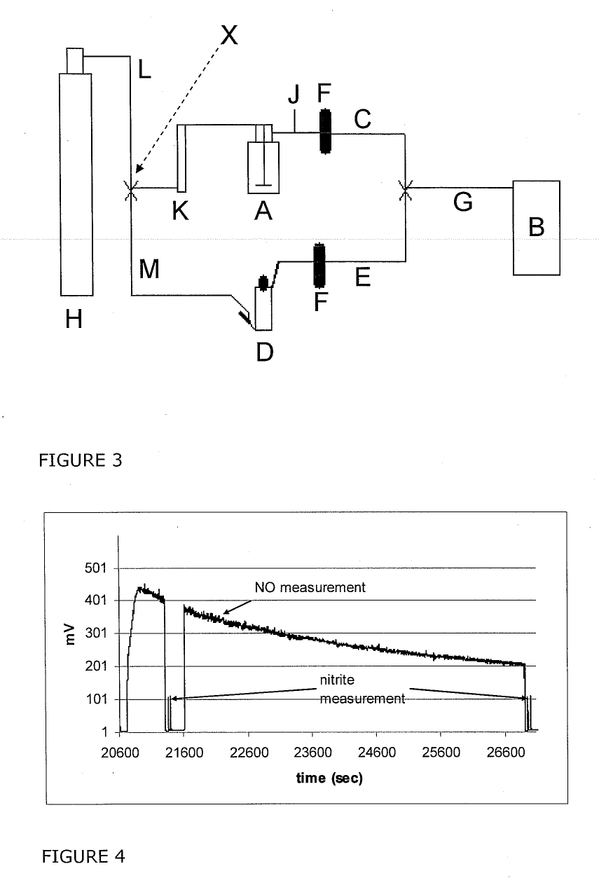

FIG. 3 illustrates a system according to the invention wherein the first vessel (A) and purge vessel (D) are connected in parallel.

FIG. 4 illustrates monitoring of NO release and NO₂⁻ background in a single analytical run.

In an embodiment, the present invention provides a method for combined measurement of release of nitrite (NO₂⁻) and nitric oxide (NO) from a probe containing nitric oxide, comprising the steps of:
placing the probe in a first vessel, in which nitrite (NO₂⁻) and nitric oxide (NO) is released from the probe;
conveying nitric oxide (NO) released directly from the probe through a first conduit to a gas feed conduit, the gas feed conduit being arranged to convey nitric oxide into a nitric oxide analysis apparatus;
removing nitrite (NO₂⁻) from the first vessel;
transforming the removed nitrite (NO₂⁻) to nitric oxide (NO) to obtain nitrite-derived nitric oxide, the step of transforming occurring subsequent to the step of removing nitrite from the first vessel;
conveying the nitrite-derived nitric oxide through a second conduit to the gas feed conduit;
operating a selector valve arranged at an upstream end of the gas feed conduit and at respective downstream ends of the first and second conduits, so as to selectively allow one of the directly released nitric oxide (NO) and the nitrite-derived nitric oxide (NO) into the gas feed conduit.

The measurement of both nitrite and nitric oxide can, using the methods of the present invention, be performed in a single analytical run.

In an embodiment, the invention provides a system for measurement of release of nitrite and nitric oxide from a probe containing nitric oxide, the system comprising:
a first vessel for accommodation of the probe;
a nitric oxide analysis apparatus;
a gas feed conduit, through which nitric oxide may be conveyed to said analysis apparatus;
a first conduit, through which nitric oxide released directly from the probe may be conveyed from the first vessel to an upstream end of the gas feed conduit;
a purge vessel arranged upstream of a second conduit, whereby nitrite may be transformed to nitric oxide in the purge vessel, the second conduit being arranged to convey nitrite-derived (may be conveyed) to the upstream end of the gas feed conduit;
a selector valve arranged at the upstream end of the gas feed conduit, the selector valve being connected to respective downstream ends of the first and second conduits, and arranged to selectively allow directly released nitric oxide and nitrite-derived nitric oxide into the gas feed conduit.

### Dynamic Head Space

In a presently preferred embodiment of the invention, on-line detection of NO release is carried out in a dynamic head space chamber containing a defined solution, such as an aqueous solution, and a NO releasing probe. The head space chamber is continuously flushed with a controlled flow of a suitably high grade inert gas, such as an inert gas selected form the group consisting of nitrogen, argon, and helium, preferably nitrogen.

A flow controller may be used to ensure a uniform flow of the inert gas. In addition or alternatively, a flow meter may be used to allow the fluctuation in the inert gas pressure/flow to be considered when calculating the concentration of NO/NO₂⁻. The flow controller/meter may therefore be connected to the NO analysis apparatus to allow for this calculation to be performed, e.g. by computer.

With respect to the examples (as shown in the figures) it is apparent that other inert gases may be used in place of nitrogen. In this context an inert gas is a gas which prevents or reduces the oxidation of the NO, typically by displacing oxygen.

The inert gas flowing through the head space chamber ensures an oxygen free solution in the head space chamber, avoiding transformation of NO into NO₂⁻. Additionally, the inert gas flushing the head space chamber strips of any NO released into the solution in the head space chamber and carries the released NO to the NO analyzer. The gas flow into the NO analyzer is controlled by a static frit restrictor mounted at the inlet of the analyzer while inert gas flow into the head space chamber is controlled by an adjustable flow controller. The inert gas flow into the head space chamber is adjusted to a higher flow than the flow into the NO analyzer, ensuring no leakage of NO or O₂ from the outside atmosphere to the head space chamber. The excess gas volume is exhausted through a vent, such as a T connection, mounted downstream of the head space chamber. This split flow system can additionally be used for regulating the sensitivity of the NO analyzer. In cases where NO release in the head space vessel exceeds detection range of the NO analyzer, the signal can be decreased by increasing the inert gas flow into the head space vessel keeping the flow into the analyzer substantially constant. This manoeuvre decreases the fraction of the NO in the inert gas stream that enters the detector, resulting in a decreased signal at the detector.

A filter, such as a hydrophobic filter, may be placed between the head space chamber and the NO analyser to prevent contamination, such as water vapour, from passing from the head space chamber to the NO analyser. Suitably, such a filter may also be placed between the purge vessel and the NO analyser.

### Nitrite Detection Unit

In a preferred embodiment, the measurement of nitrite is carried out in a purge vessel system continuously flushed with a controlled flow of inert gas, preferably nitrogen (N₂), to ensure oxygen free environment in the purge vessel. The vessel contains an acidic solution of sodium iodide allowing the following reaction to take place upon injection of a nitrite containing sample:

I⁻+NO₂⁻+2H⁺->NO+½I₂+H₂O

Upon reduction of NO₂⁻ to NO, NO is drawn into the analyzer and measured as NO gas in the same detection system as used for dynamic head space measurement.

Suitably the step of transforming nitrite to nitric oxide may be carried out using a suitable reducing agent, such as sodium iodide or potassium iodide.

### Combined on Line NO Release and NO₂⁻ Measurements

Since NO₂⁻ is an unwanted side product in most NO release systems, measuring the combined NO release and NO₂⁻ concentration in these systems is of significant importance. In the method and system of the present invention, NO release is, suitably, measured in e.g. a dynamic head space chamber setup while nitrite is measured in a purge vessel setup. The two setups are combined in a selector valve, such as a 4-way stop cock, enabling the inlet to the NO analyzer to be switched between dynamic head space and purge vessel setup. By switching from dynamic head space to purge vessel setup during a NO measurement and withdrawing a sample, preferably a liquid sample, from the head space chamber and injecting it into the purge vessel system it is possible to monitor both NO release and NO₂⁻ background in a single analytical run as shown in FIG. 4. Following NO gas and liquid nitrite calibrations, the mV signal obtained from the analyzer can be transformed into actual concentrations. The present invention therefore provides a method, and a system, for independent analysis of both NO and nitrite from a single combined analysis. As shown in FIGS. 1 and 3 the first vessel and purge vessel may be arranged either in series (FIG. 1) or in parallel (FIG. 3).

### FURTHER FEATURES OF THE INVENTION

The step of conveying nitric oxide (NO) may comprise continuous transport of directly released nitric oxide away from the first vessel. During such transport, the selector valve should preferably be in a position, in which nitric oxide is allowed into the analysis apparatus. Nitrogen (N₂), or an alternative inert gas as referred to herein, may be used as a carrier gas for transport of nitric oxide (NO). A first gas feed conduit may be provided, through which the inert gas may be conveyed into the first vessel. A pressure tank containing the inert gas may be used as a gas source, whereby excess pressure in the pressure tank provides a pressure gradient in the system, which ensures appropriate transport of gasses. The flow rate of inert gas in the gas feed conduit may be measured by means of a flow meter arranged in the nitrogen feed conduit, the flow meter producing an output signal, which is passed to the analysis apparatus.

The first vessel may comprise a continuously flushed headspace chamber, in which case the nitric oxide analysis apparatus may continuously analyse gas fed to the apparatus via the gas feed conduit.

Typically, the probe is placed within a liquid solvent within said first vessel. The solvent is preferably a solvent which is capable of inducing the release of nitric oxide (and suitably nitrite) from the probe and/or transporting the nitric oxide (an suitably nitrite) released from the probe.

The sample, as referred to in the method for the measurement of the release of nitrite (NO₂⁻) and nitric oxide from a sample, may be prepared by the placing of the probe containing nitric oxide, for example in the form of a nitric oxide adduct, within a solvent in a first vessel (A), which nitrite (NO₂⁻) and nitric oxide (NO) is released from the probe into the solvent. The first fraction may therefore be the sample, and the second fraction a portion of the sample removed from the first fraction/sample for conversion of the nitrite to nitric oxide. The nitric oxide (NO) released from the sample (or first fraction) may be passed through a first conduit (C) to a gas feed conduit (G), the gas feed conduit being arranged to convey nitric oxide into a nitric oxide analysis apparatus (B). The second sample may be a nitrite (NO₂⁻) sample removed from the first vessel (A).

In one embodiment, therefore, the sample, as referred to in the method for the measurement of the release of nitrite (NO₂⁻) and nitric oxide from a sample is typically the solvent which has been exposed to a probe.

In one embodiment, the first fraction, referred to in the method for the measurement of the release of nitrite (NO₂⁻) and nitric oxide from a sample, is typically the solvent, or a portion thereof, which has been exposed to the probe.

In one embodiment, the second fraction, referred to in the method for the measurement of the release of nitrite (NO₂⁻) and nitric oxide from a sample, is typically the nitrite sample.

Therefore, the sample, as referred to in the method for the measurement of the release of nitrite (NO₂⁻) and nitric oxide from a sample, and the first and second fraction typically originate form a single source and are equivalent. They typically comprise an equivalent concentration of NO and/or NO₂⁻.

Therefore, it is envisaged that, in one embodiment, the at least two equivalent fractions of a sample comprising a combined mixture of nitrite and nitric oxide, refer to the solvent which has been exposed to the probe in the first vessel (first fraction), and a nitrite sample which is a fraction of the solvent that has been subsequently removed from the first vessel (second fraction). The conversion of the nitrite component of a second fraction to nitric oxide may therefore occur within the purge vessel. Suitably the nitric oxide analysis apparatus is used for the determination of the concentration of nitric oxide in (or released by) the first and second fractions (after conversion of the nitrite component of a second fraction to nitric oxide).

In a preferred embodiment, the probe is immersed in an aqueous solution. The aqueous solution may be a solution mimicking physiological solution such as e.g. blood, isotonic salt water or buffers adjusted to physiological pH, salt concentration and surface tension such as e.g. a pH adjusted PBS buffer containing tween 20 to adjust surface tension. The aqueous solution may be optimized to increase nitric oxide release from the probe, e.g. by optimizing the pH to a given release optimum or by adding any nitric oxide release activators to the solution in the head space chamber.

It will be apparent that the aqueous solution may be substituted partially or entirely with an alternative solvent, such as an alternative polar solvent, for example an alcohol such as methanol or ethanol.

In one preferred embodiment, the solvent, such as aqueous solution or alternative solvent, is selected for its ability to induce NO release from the probe, such as from one or more of the NO adducts as referred to herein.

Indeed, in one specific embodiment, it is envisaged that the probe need not necessarily be inserted into an aqueous or liquid solvent, but within a gaseous phase, such as within the inert gas referred to herein. Therefore, in one embodiment the probe may be mounted in a (free) gas or (free) gas stream (e.g. in the stream of the inert gas), this embodiment can be used to measure, for example, spontaneous nitric oxide/nitrite release from the probe.

In one embodiment, the gas phase or stream may comprise a vapor, for example using the partial pressure of the vapor may be used to activate the release of nitric oxide from the probe. The vapor may, in one embodiment be water vapor, or the vapor of an alternative solvent as referred to herein.

In one embodiment, the solvent is essentially free from molecular oxygen, i.e. the levels of O₂ do not adversely affect the accuracy of the measurement of NO and nitrite release from the probe.

The probe may comprise any material which is capable of releasing nitric oxide, and preferably nitrite, either spontaneously or in the presence of an activator.

The probe may be a gas, although more suitable the probe is a liquid or a solid, and preferably the solvent is a liquid.

In a preferably embodiment, the probe is a solid.

In a specific embodiment the probe is a medical device, or a coating, such as an NO adduct coating, on a medical device, such as an intravascular medical device.

With regard to analyzing of NO and NO₂⁻ release from medical devices, in particular implants, it may be desired that the conditions of the aqueous solution resemble the physiological conditions of the human or animal body in the best possible way. For example, the viscosity of the solution may be close to that of blood, and the solution may be maintained at body temperature, i.e. at approximately 37° C. In one embodiment the aqueous solution is an ionic solution. A pressure above atmospheric pressure may be maintained in the first conduit in order to prevent atmospheric air from entering the system. Thus, in particular ingress of oxygen may be prevented. Excess gas in flow in the first conduit may be released through a vent in the first conduit arranged downstream of the first vessel (or head space chamber) and preferably upstream of the selector valve.

The step of conveying nitrite (NO₂⁻) may comprise sampling at least one nitrite sample from the first vessel and conveying the sample to the second conduit, following transformation into nitric oxide. Transformation may e.g. occur in the purge vessel. The nitrite sample may be conveyed into the purge vessel arranged upstream of the second conduit. A suitable inert gas, preferably nitrogen (N₂), may be used as a carrier gas for transport of nitrite derived nitric oxide from the purge vessel through the second conduit and the gas feed conduit. In one embodiment, the nitrite sample is conveyed manually from the first vessel to the purge vessel. In another embodiment, transport of the nitrite sample is effected by an auto-sampling system. In such an embodiment, operation of the auto-sampling system is preferably synchronized with operation of the selector valve, so that the nitrite sample is only conveyed into the second conduit when the selector valve is in a position allowing the flow of nitrite derived nitric oxide into the analysis apparatus.

The probe may comprise a medical device, such as an intermittent or permanent intravascular implant, such as a stent, a stent graft, a balloon, a balloon catheter, a guidewire, an introducer sheath, or an embolization device. The medical device may incorporate or be coated with nitric oxide or with a coating material, such as a suitable polymer, loaded with NO or a NO-releasing agent (an NO adduct).

The probe preferably comprises a NO adduct, i.e. a substance which gives off NO as a result of a chemical reaction when wetted or exposed to enzymes or other chemicals. NO adducts are therefore considered to be compounds which can store NO.

The nitric oxide adducts may be monomers of polymers, and may be selected from compounds which, for example, comprise nitrosyl, nitrite, nitrate, nitroso, nitrosothio, nitro, metal-NO complex, nitrosamine, nitrosimine, diazetine dioxide, furoxan, benzofuroxan or NONOate (—N₂O₂⁻) groups.

The nitric oxide adduct preferably comprises a nitric oxide-nucleophile complexes. The nitric oxide-adduct may be monomer or a polymer.

Nitric oxide adducts which are monomeric molecules may be soluble or insoluble in physiological media. Suitable monomeric nitric oxide adducts are, for example, disclosed in U.S. Pat. No. 4,954,526.

Numerous polymers which are capable of releasing nitric oxide in physiologic media are known in the art. For example, the polymers disclosed in U.S. Pat. No. 5,405,919 and U.S. Pat. No. 6,875,840 may be used.

It is preferable that the nitric oxide adduct is in the form of a polymer, such as a linear polymer, a branched polymer, and/or a cross linked polymer, to which is bound a nitric oxide releasing functional group, such as a nitric oxide-nucleophile complexes.

In one embodiment, the nitric oxide adduct is selected from the group consisting of: nitroglycerin, sodium nitroprusside, S-nitroso-proteins, S-nitrosothiols, long carbon-chain lipophilic S-nitrosothiols, S-nitroso-dithiols, iron-nitrosyl compounds, thionitrates, thionitrites, sydnonimines, furoxans, organic nitrates, and nitrosated amino acids. nitroso-Nacetylcysteine, S-nitroso-captopril, S-nitroso-homocysteine, S-nitroso-cysteine, S-nitroso-glutathione, and S-nitrosopenicillamine, S-nitrosothiols, S-nitrosylated polysaccharides such as S-nitrosylated cyclodexrins, NONOate compounds (i.e. compounds which comprise the anionic NONOate functional group (N₂O₂⁻)), NONOate polymers.

It is recognised that some NO adducts are water inducible, i.e. they accept protons from ionic water, which results in the release of nitric oxide (e.g. NONOates). It is preferable that such water inducible NO adducts are used.

However, it is also envisaged that other NO adducts may also be employed. For example enzymatic release of NO may also be utilised by incorporation of suitable enzymes into the nitric oxide adduct layer. In such an embodiment, the aqueous solution may comprise an enzyme capable of acting on the NO adduct to release NO. The enzyme may for example be NO synthase.

In one embodiment the nitric oxide adduct is a NONOate, such as a polymeric NONOate selected from the group consisting of: polyolefins, such as polystyrene, polypropylene, polyethylene, polytetrafluorethylene, polyvinylidene difluoride, polyvinylchloride, derivatized polyolefins such as polyethylenimine, polyethers, polyesters, polyamides such as nylon, polyurethanes, biopolymers such as peptides, proteins, oligonucleotides, antibodies and nucleic acids, starburst dendrimers.

A most preferred nitric oxide adduct polymer is polyethylenimine diazeniumdiolate, such as linear polyethylenimine diazeniumdiolate (LPEI-NONO).

In the embodiment of FIG. 2, no conduit is provided to connect the head space chamber with the purge vessel. It is contemplated that, in this embodiment, nitrite will be conveyed by manual means.

In FIG. 3, the headspace chamber and purge vessel are arranged in parallel, allowing a single source of inert gas (such as nitrogen) to be employed. In some embodiments, such a parallel arrangement is preferred.

1. A method for measurement of the release of nitrite (NO₂⁻) and nitric oxide (NO) from a probe containing nitric oxide or a nitric oxide adduct, comprising the steps of:
placing the probe in a first vessel (A), in which nitrite (NO₂⁻) and nitric oxide (NO) is released from the probe;
conveying nitric oxide (NO) released from the probe through a first conduit (C) to a gas feed conduit (G), the gas feed conduit being arranged to convey nitric oxide into a nitric oxide analysis apparatus (B);
removing a nitrite (NO₂⁻) sample from the first vessel (A);
transforming the removed nitrite (NO₂⁻) in the nitrite sample to nitric oxide (NO) to obtain nitrite-derived nitric oxide, the step of transforming occurring subsequent to the step of removing nitrite from the first vessel (A);
conveying the nitrite-derived nitric oxide through a second conduit (E) to the gas feed conduit (G);
operating a selector valve (X) arranged at an upstream end of the gas feed conduit (G) and at respective downstream ends of the first (C) and second (E) conduits, so as to selectively allow one of the directly released nitric oxide (NO) and the nitrite-derived nitric oxide (NO) into the gas feed conduit (G).

2. The method according to claim 1, wherein a further step of determining the amount of directly released nitric oxide (NO) and nitrite derived nitric oxide (NO) from the probe.

3. The method according to claim 1, wherein the step of conveying directly released nitric oxide (NO) comprises continuous transport of nitric oxide away from the first vessel (A) when the selector valve (X) is in a position, in which nitric oxide is allowed into the analysis apparatus (B).

4. The method according to claim 1, wherein an inert gas is used as a carrier gas for transport of directly released nitric oxide (NO).

5. The method according to claim 4, wherein the inert gas is nitrogen (N₂).

6. The method according to claim 4 further comprising a first inert gas feed conduit (L), through which the inert gas (H) is conveyed into the first vessel.

7. The method according to claim 6, wherein the flow rate of inert gas in the inert gas feed conduit (L) is measured by means of a flow meter (K) arranged in the inert gas feed conduit (L), the flow meter producing an output signal, which is passed to the analysis apparatus (B).

8. The method according to claim 1, wherein the first vessel comprises a continuously flushed headspace chamber.

9. The method according to claim 1, wherein the probe is placed within a liquid solvent within said first vessel.

10. The method according to claim 9 wherein the solvent is an aqueous solution.

11. The method according to claim 9, wherein the solvent is maintained at a temperature of approximately 37° C.

12. The method according to claim 1, wherein a pressure above atmospheric pressure is maintained in the first conduit.

13. The method according to claim 12, further comprising the step of releasing a portion of the gas flow in the first conduit (C) through a vent in the first conduit (J).

14. The method according to any claim 1, wherein the step of conveying nitrite (NO₂⁻) comprises the step of sampling at least one nitrite sample from the first vessel (A) and conveying the sample to a purge vessel (D) arranged upstream of the second conduit, and wherein the step of transforming nitrite to nitric oxide is carried out in said purge vessel (D).

15. The method according to claim 14, wherein the nitrite (NO₂⁻) sample removed from the first vessel is a fraction of the solvent which has been exposed to the probe.

16. The method according to claim 14, wherein the step of transforming nitrite to nitric oxide is carried out using a reducing agent.

17. The method according to claim 16, wherein the reducing agent is sodium iodide or potassium iodide.

18. The method according to claim 14, wherein an inert gas (H) is used as a carrier gas for transport of nitrite derived nitric oxide from the purge vessel (D) through the second conduit (E) to the gas feed conduit (G).

19. The method according to claim 18, wherein the inert gas is nitrogen (N₂).

20. The method according to claim 14, wherein the nitrite sample is conveyed manually from the first vessel.

21. The method according to claim 14, wherein transport of the nitrite sample is effected by an auto-sampling system, and wherein operation of the pump system is synchronized with operation of the selector valve (X).

22. The method according to claim 1, wherein the release of nitric oxide form the probe occurs spontaneously.

23. The method according to claim 1, wherein the release of nitric oxide is achieved by the addition of an activator within the first vessel.

24. The method according to claim 1, wherein the probe is a liquid capable of releasing nitric oxide.

25. The method according to claim 1, wherein the probe is a solid capable of releasing nitric oxide.

26. The method according to claim 1 wherein the probe comprises a medical device.

27. The method according to claim 26, wherein the medical device comprises an intermittent or permanent intravascular implant.

28. The method according to claim 27, wherein the medical device is selected from the group consisting of: a stent, a stent graft, a balloon, a balloon catheter, a guidewire, an introducer sheath and an embolization device.

29. The method according to claim 1, wherein the probe comprises an NO adduct.

30. The method according to claim 29, wherein the NO adduct is polyethylenimine dizeniumdiolate.

31. The method according to claim 1, wherein the concentration of nitric oxide in the NO analyzer apparatus is determined by a gas phase chemiluminescence reaction between nitric oxide and ozone.

32. The method according to claim 31, wherein the chemiluminescence is detected by a photomultiplier tube.

33. The method according to claim 32, wherein the sensitivity of the photomultiplier tube is controlled by regulation of the voltage supplied to the photomultiplier tube.

34. A system for measurement of release of nitrite and nitric oxide from a probe containing nitric oxide or a nitric oxide adduct, the system comprising:
a first vessel (A) for accommodation of the probe;
a nitric oxide analysis apparatus (B);
a gas feed conduit (G), through which nitric oxide may be conveyed to said analysis apparatus;
a first conduit (C), through which nitric oxide released directly from the probe may be conveyed from the first vessel (A) to an upstream end of the gas feed conduit (G);
a purge vessel (D) arranged upstream of a second conduit (E), whereby nitrite may be transformed to nitric oxide in the purge vessel (D), the second conduit (E) being arranged to convey the nitrite derived nitric oxide from the purge vessel (D) to the upstream end of the gas feed conduit (G);
a selector valve (X) arranged at the upstream end of the gas feed conduit (G), the selector valve being connected to respective downstream ends of the first (C) and second (E) conduits, and arranged to selectively allow directly released nitric oxide and nitrite-derived nitric oxide into the gas feed conduit (G).

35. A system according to claim 34, wherein the system further comprises a source of inert gas (H) which is connected to the first vessel (D) by a first inert gas feed conduit so that the inert gas is capable of conveying the nitric oxide released from the probe from the first vessel (A) to the upstream end of the gas conduit (G).

36. A system according to claim 35, wherein the system further comprises a source of inert gas (H) which is connected to the purge vessel (D) by a second inert gas feed conduit (M) so that the inert gas is capable of conveying the nitrite-derived nitric oxide from the purge vessel (D) to the upstream end of the gas conduit (G).

37. A system according to claim 35 wherein a flow meter and/or a flow controller (K) is placed within the first inert gas feed conduit (G).

38. The system according to claim 37 wherein the flow meter and/or a flow controller (K) is connected to the analysis apparatus as to allow the gas flow level to be considered in the calculation of nitric oxide and nitrite levels.

39. A system according to claim 37, wherein there is a common inert gas source (H) connected to the upstream ends of both the first and second inert gas feed conduits (L and M).

40. A system according to claim 34 wherein the first vessel (A) and purge vessel (D) are arranged in parallel.

41. A system according to claim 34 wherein the first vessel (A) and purge vessel (D) are arranged in series.

42. A system according to claim 34, wherein the first vessel (A) comprises a gaseous or liquid solvent capable of transferring nitric oxide from the probe to the gas feed conduit (G).

43. The system according to claim 42, wherein the solvent is a liquid solvent or an aqueous solution.

44. The system according to claim 34 wherein the first conduit (C) comprises a filter (F).

45. The system according to claim 34, wherein said second conduit (E) comprises a filter (F).

46. The system according to claim 34 wherein said first conduit comprises a vent (J).

47. The system according to claim 34 wherein said first vessel is a continuously flushed headspace chamber.

48. The system according to claim 34, wherein the NO analyzer apparatus is capable of qualitative detection of the light emitted by a gas phase chemiluminescence reaction between nitric oxide and ozone.

49. The system according to claim 48, wherein the quantitative detection of chemiluminescence is detected by a photomultiplier tube.

50. The system according to claim 49, wherein the photomultiplier tube is controlled by regulation of the voltage supplied to the photomultiplier tube.

51. A method for the measurement of the release of nitrite (NO₂⁻) and nitric oxide from a sample, comprising the following steps:
a) Obtaining at least two equivalent fractions of a sample comprising a combined mixture of nitrite and nitric oxide;
c) determining the concentration of nitric oxide released from a first fraction;
d) converting the nitrite component of a second fraction to nitric oxide;
e) determining the concentration of nitric oxide released from the second fraction after conversion of nitrite to nitric oxide;
f) comparing the levels of nitric oxide determined in step (c) and step (e) to determine the concentration of nitrite (NO₂⁻) and nitric oxide in the sample
wherein steps c) and d) may be carried out in any order.

52. The method according to claim 51, wherein the at least two equivalent fractions are obtained from a single analytical procedure from the same sample.

53. The method according to claim 52, wherein the first and second fractions are obtained by separating the second fraction from the sample to leave the first fraction.

54. The method according to claim 52, wherein the at least two equivalent fractions are obtained as consecutive fractions from a single analytical procedure.

55. The method according to claim 51, wherein the sample is obtained from a nitric oxide adduct.

56. The method according to claim 55, wherein the nitric oxide adduct is polyethylenimine diazeniumdiolate.

57. The method according to claim 56, wherein the sample is obtained by the release of nitrite and nitric oxide from a nitric oxide adduct coating applied to a medical device.

58. The method according to claim 57, wherein the medical device comprises an intermittent or permanent intravascular implant.

59. The method according to claim 58, wherein the medical device is selected from the group consisting of: a stent, a stent graft, a balloon, a balloon catheter, a guidewire, an introducer sheath and an embolization device.

60. The method according to claim 51, wherein the step of transforming nitrite to nitric oxide is carried out using a reducing agent.

61. The method according to claim 60, wherein the reducing agent is sodium iodide or potassium iodide.

62. The method according to claim 51, wherein the concentration of nitric oxide is determined by a gas phase chemiluminescence reaction between nitric oxide and ozone.

63. The method according to claim 62, wherein the chemiluminescence is detected by a photomultiplier tube.

64. The method according to claim 63, wherein the sensitivity of the photomultiplier tube is controlled by regulation of the voltage supplied to the photomultiplier tube.
